(19) United States
(12) Patent Application Publication
(10) Pub. No.: US 2003/0093022 A1
(43) Pub. Date: May 15, 2003

(54) Back stress relief system

(76) Inventor: Paul Sund, Newburg, ND (US)

Correspondence Address: Michael S. Neustel, 2534 South University Drive, Suite No. 4, Fargo, ND, 58103, US

(21) Appl. No.: 10/011,188

(22) Filed: Nov. 13, 2001

(51) Int. Cl. A61F 5/00

(52) U.S. Cl. 602/33

(57) ABSTRACT

A back stress relief system for alleviating stress and pain within an individual's back. The back stress relief system includes a support frame, a platform attached to the support frame, an opening within the platform, a first extended member and a second extended member extending upwardly from the platform, a sliding member slidably positioned within the support frame below the opening, a pair of leg cuffs attached to the sliding member and extending through the opening, and a drive unit mechanically attached to the sliding member for manipulating the sliding member along a longitudinal axis of the support frame. An individual lays flat upon the platform and positions the extended members within the axilla defined by the junction between the arm and shoulder. The legs of the individual are secured within the leg cuffs and then the drive unit is operated to extend the legs away from the extended members thereby alleviating stress within the back and joints of the individual.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to back assistance devices and more specifically it relates to a back stress relief system for alleviating stress and pain within an individual's back.

Back problems are the most common physical complaint among American adults. Nonspecific back pain is a leading cause of lost job time, to say nothing of the time and money spent in search of relief. The spinal column is an extraordinary mechanism, providing the stability we use to stand upright and the flexibility we need for active movement. The spine, or backbone, is actually a stack of 24 individual bones called vertebrae. A healthy spine is S-shaped when viewed from the side, curving back at the shoulders and inward at the neck and small of the back. In addition to serving as the body's main structural member, it also houses the spinal cord—the intricate sensory network that runs through the vertebrae to transmit feeling and control movement throughout the entire body.

The main reason late-20th-century Americans suffer from back problems is that we are increasingly defying our evolutionary heritage and becoming sedentary creatures. The upright posture is designed for walking, and that's what people did for most of human history. Only in this century, with the advent of motor vehicles, have most people gone from place to place while sitting down. In much the same way, our work habits are changing. Most of our ancestors worked standing up—hunting, gathering, and working on farms or at workbenches. Today, a high proportion of people spend the better part of their working day sitting at desks, at workstations, or in cars and trucks. These recent but momentous changes in human behavior have had a profound—and largely negative—impact on human physiology. People who walk a lot or do physical labor develop good muscle tone in their backs and legs. People who sit most of the day lose that muscle tone, and their backs are the first place to show it.

2. Description of the Prior Art

Back support devices have been in use for years for relieving pain. Conventional back support devices are comprised of a belt-like structure that surrounds an individuals waste for applying inward pressure upon an individual's back. There are numerous other back pain relief devices on the mark, however, none of these devices are designed for alleviating the stress applied to a back.

Examples of patented devices which are related to the present invention include U.S. Pat. No. 6,045,525 to Chitwood; U.S. Pat. No. 4,995,378 to Dyer et al.; U.S. Pat. No. 6,039,737 to Dyer; U.S. Pat. No. 3,238,936 to Siedentop; U.S. Pat. No. 2,865,367 to Sorenson; U.S. Pat. No. 2,856,917 to Mack; and U.S. Pat. No. 4,378,791 to Sarrell.

While these devices may be suitable for the particular purpose to which they address, they are not as suitable for alleviating stress and pain within an individual's back. Conventional back support devices are not designed for relieving stress upon an individual's back.

In these respects, the back stress relief system according to the present invention substantially departs from the conventional concepts and designs of the prior art, and in so doing provides an apparatus primarily developed for the purpose of alleviating stress and pain within an individual's back.

### SUMMARY OF THE INVENTION

In view of the foregoing disadvantages inherent in the known types of back pain relief devices now present in the prior art, the present invention provides a new back stress relief system construction wherein the same can be utilized for alleviating stress and pain within an individual's back.

The general purpose of the present invention, which will be described subsequently in greater detail, is to provide a new back stress relief system that has many of the advantages of the back pain relief devices mentioned heretofore and many novel features that result in a new back stress relief system which is not anticipated, rendered obvious, suggested, or even implied by any of the prior art back pain relief devices, either alone or in any combination thereof.

To attain this, the present invention generally comprises a support frame, a platform attached to the support frame, an opening within the platform, a first extended member and a second extended member extending upwardly from the platform, a sliding member slidably positioned within the support frame below the opening, a pair of leg cuffs attached to the sliding member and extending through the opening, and a drive unit mechanically attached to the sliding member for manipulating the sliding member along a longitudinal axis of the support frame. An individual lays flat upon the platform and positions the extended members within the axilla defined by the junction between the arm and shoulder. The legs of the individual are secured within the leg cuffs and then the drive unit is operated to extend the legs away from the extended members thereby alleviating stress within the back and joints of the individual.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a back stress relief system that will overcome the shortcomings of the prior art devices.

A second object is to provide a back stress relief system for alleviating stress and pain within an individual's back.

Another object is to provide a back stress relief system that may be utilized by individuals of various sizes and lengths.

An additional object is to provide a back stress relief system that is easy to utilize.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is an exploded upper perspective view of the present invention.

FIG. 3 is a side view of a first alternative embodiment of the present invention.

FIG. 4 is an upper perspective view of the first alternative embodiment with the platform removed.

FIG. 5 is an upper perspective view of a second alternative embodiment with the platform removed.

### DESCRIPTION OF THE PREFERRED EMBODIMENT

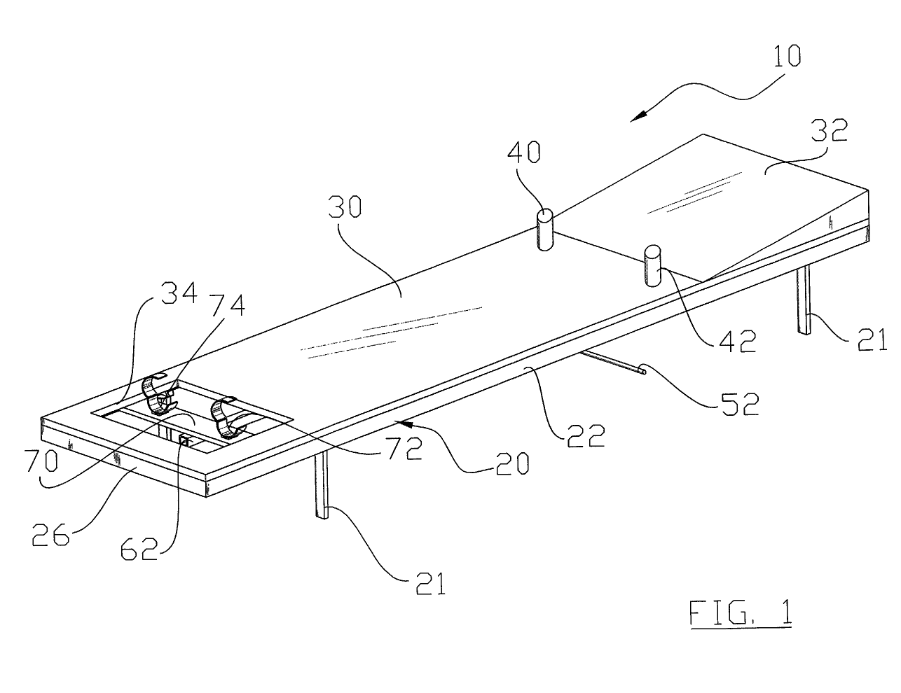

Turning now descriptively to the drawings, in which similar reference characters denote similar elements throughout the several views, FIGS. 1 through 5 illustrate a back stress relief system 10, which comprises a support frame 20, a platform 30 attached to the support frame 20, an opening 34 within the platform 30, a first extended member 40 and a second extended member 42 extending upwardly from the platform 30, a sliding member 70 slidably positioned within the support frame 20 below the opening 34, a pair of leg cuffs 72, 74 attached to the sliding member 70 and extending through the opening 34, and a drive unit 50 mechanically attached to the sliding member 70 for manipulating the sliding member 70 along a longitudinal axis of the support frame 20. An individual lays flat upon the platform 30 and positions the extended members 40, 42 within the axilla defined by the junction between the arm and shoulder. The legs 21 of the individual are secured within the leg cuffs 72, 74 and then the drive unit is operated to extend the legs 21 away from the extended members 40, 42 thereby alleviating stress within the back and joints of the individual.

As shown in FIGS. 1 through 5 of the drawings, the support frame 20 is preferably formed into an elongate structure. The support frame 20 preferably is at least fix feet in length and at least two feet in width, though greater or less dimensions may be utilized for accommodating and supporting a human body. The support frame 20 may be comprised of various types of materials and structures as can be appreciated.

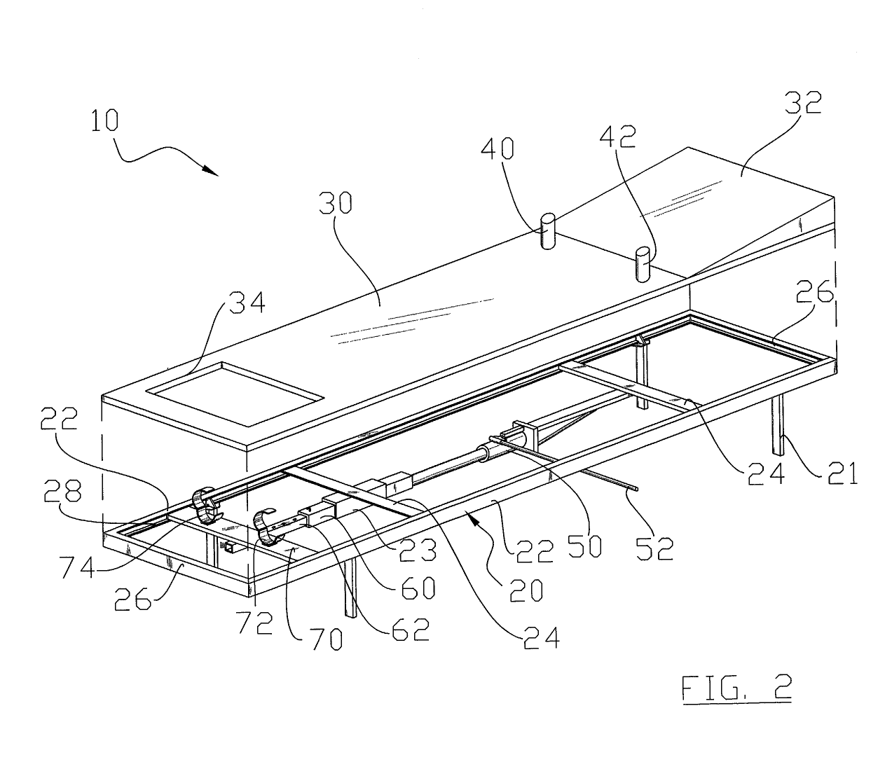

As shown in FIGS. 2, 4 and 5 of the drawings, the support frame 20 may be comprised of a pair of side members 22, a pair of end members 26 attached between the side members 22, and a plurality of legs 21 extending downwardly for supporting the platform 30 at an elevated height. At least one cross member 24 is preferably attached between the side members 22 as shown in FIGS. 2, 4 and 5 of the drawings.

As shown in FIGS. 2, 4 and 5 of the drawings, the side members 22 include an inner lip portion 28 for slidably supporting the sliding member 70. Various other structures may be utilized within the support frame 20 for slidably supporting the sliding member 70.

As shown in FIGS. 1 through 3 of the drawings, the platform 30 is a generally flat structure formed for supporting an individual lying upon their back. The platform 30 may included an elevated headrest 32 for comfortably supporting an individual's head as shown in FIGS. 1 through 3 of the drawings. The platform 30 may be comprised of various types of materials and structures.

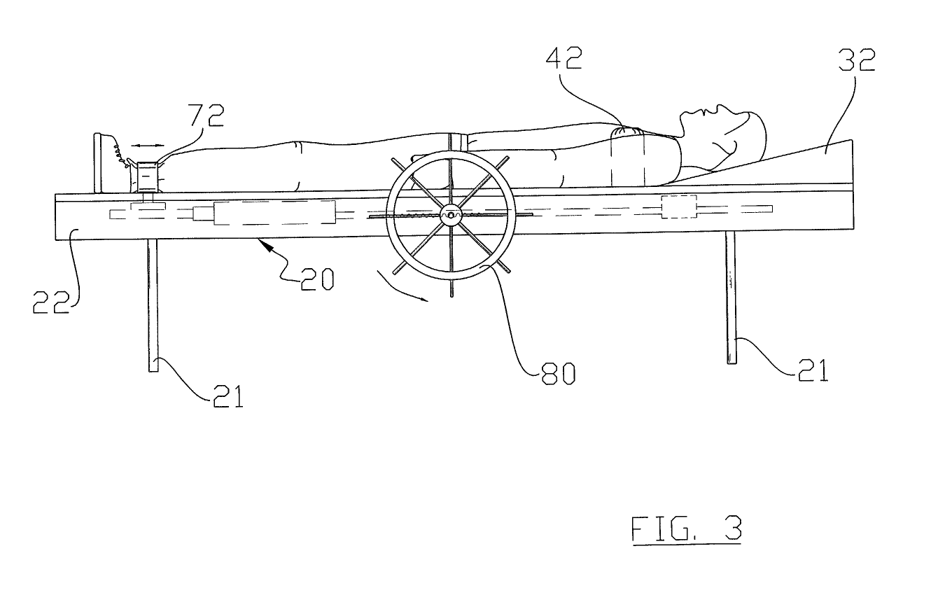

As shown in FIGS. 1 and 2 of the drawings, a first extended member 40 and a second extended member 42 extend upwardly from the upper surface of the platform 30. The extended members 40, 42 may be comprised of a rigid structure with a cushion surrounding the exterior portion thereof. It can be appreciated that the extended members 40, 42 may be inserted into various locations along the length of the platform 30 for accommodating individuals of various heights. When an individual lays flat upon the platform 30, the individual positions the extended members within the axilla defined by the junction between the arm and shoulder as shown in FIG. 3 of the drawings.

As shown in FIGS. 1 and 2 of the drawings, the platform 30 includes an opening 34 that receives a first leg cuff 72 and a second leg cuff 74 extending from the sliding member 70. The opening 34 may have various shapes and sizes sufficient for allowing movement of the sliding member 70 in a longitudinal manner. The opening 34 is preferably sufficient in size for allowing securing of the leg cuffs 72, 74 to individuals of various heights without requiring repositioning of the extended members 40, 42. The leg cuffs 72, 74 are comprised of any structure capable of securing about the ankles of an individual such as but not limited to rigid C-shaped members or straps.

As shown in FIG. 2 of the drawings, a hydraulic drive unit 50 having a handle 52 is non-movably attached to the support frame 20 beneath the platform 30. An elongate shaft 60 extends from the hydraulic drive unit 50 and slidably through a first sleeve 23 as shown in FIG. 2 of the drawings. The first sleeve 23 may be attached to a cross member 24 or similar structure. The elongate shaft 60 may be directly connected to the sliding member 70 or an adjustable extension member 62 extending from the elongate shaft 60 may be attached to the sliding member 70. By manipulating the handle 52, the elongate shaft 60 is extended thereby extending the sliding member 70 and leg cuffs 72, 74 resulting in stress being removed from an individuals back. The first sleeve 23 guides and supports the elongate shaft 60 during the application of tension to the individual. When finished, the user simply allows the hydraulic drive unit 50 to release pressure thereby allowing the sliding member 70 to slide toward the individual.

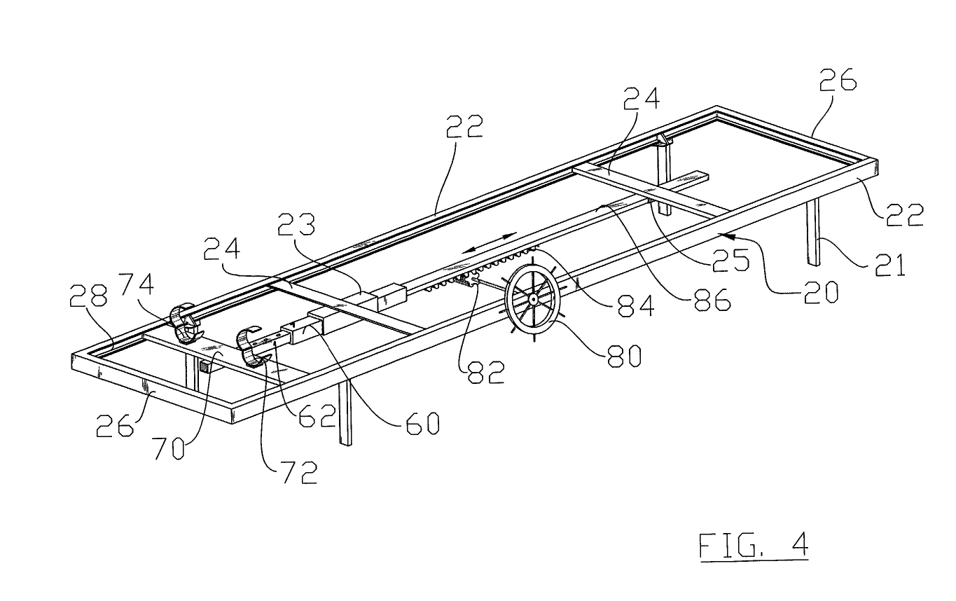

In a first alternative embodiment shown in FIGS. 3 and 4 of the drawings, a wheel member 80 is rotatably attached to the support frame 20 and mechanically engaging a first drive sprocket 82. An elongate bar member 86 attached to the elongate shaft 60 includes a plurality of gears 84 that are engaged by the first drive sprocket 82 for extending or contracting the sliding member 70. A second sleeve 25 slidably receives a portion of the elongate bar member 86 opposite of the elongate shaft 60 as shown in FIG. 4 of the drawings. The user rotates the wheel member 80 in the desired direction to move the sliding member 70 in the desired direction.

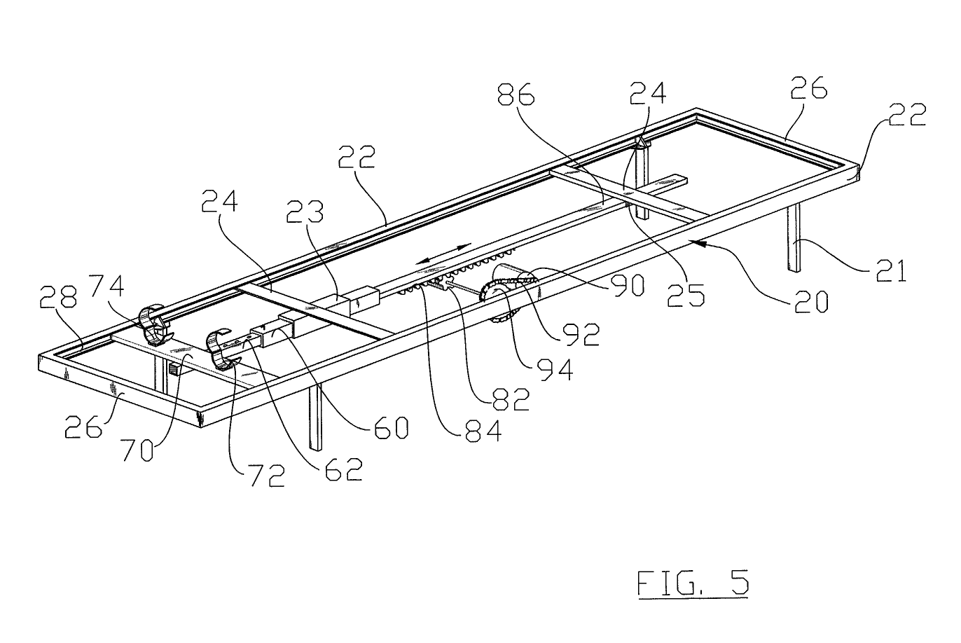

In a second alternative embodiment shown in FIG. 5 of the drawings, a drive motor 90 is attached to the support frame 20 and mechanically connected to the first drive sprocket 82 of the first alternative embodiment by the usage of a drive chain 92 and a second drive sprocket 94. The drive motor 90 may be comprised of various types of motor devices including but not limited to electric motors, hydraulic motors and the like. The drive motor 90 may be controlled utilizing any conventional control unit. The drive chain 92 may also be replaced with another suitable drive means such as but not limited to a belt.

In use, the user first positions their body upon the upper surface of the platform 30. The user positions their opposing axilla about the extended members 40, 42 in a snug manner. The user then has the ankle portions of their legs 21 secured to the leg cuffs 72, 74. Depending upon the length of the individual's body, the sliding member 70 may have to be adjusted by adjusting the extension member 62 slidably positioned within the elongate shaft 60 in an adjustable manner. Once the ankles of the user are snugly secured within the leg cuffs 72, 74, the user then operates the appropriate drive unit 50, 80 or 90 to cause the sliding member 70 to extend away from the extended members 40, 42 within the opening 34. As the sliding member 70 extends away from the individual, tension is applied to the individual's body and back thereby relieving stress from within thereof. The extended members 40, 42 prevent the upper portion of the user's body from moving thereby causing tension between the shoulders and ankles of the individual's body. When the desired amount of tension has been applied to the individual's body, the drive unit 50, 80, 90 is terminated and the position of the sliding member 70 is maintained for a desired period of time. When the individual is finished, the user simply reverses the drive unit 50, 80, 90 so that the tension is removed from their body and back. The individual is then able to remove their ankles from the leg cuffs 72, 74 and remove themselves from the platform 30.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed to be within the expertise of those skilled in the art, and all equivalent structural variations and relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A back stress relief system, comprising:
a support frame having an elongate structure;
a platform attached to said support frame having a first end and a second end;
an opening extending into said platform near said first end;
a first extended member extending upwardly from said platform near said second end;
a second extended member extending upwardly from said platform near said second end;
a sliding member slidably positioned within said support frame, wherein said sliding member is positioned below said opening within said platform;
a drive unit for manipulating said sliding member along a longitudinal axis of said support frame;
a first leg cuff attached to said sliding member, wherein said first leg cuff extends through said opening for engaging a first ankle of an individual; and
a second leg cuff attached to said sliding member, wherein said second leg cuff extends through said opening for engaging a first ankle of an individual.

2. The back stress relief system of claim 1, wherein said first extended member has a longitudinal position approximately equal to said second extended member.

3. The back stress relief system of claim 1, wherein said first leg cuff has a longitudinal position approximately equal to said second leg cuff.

4. The back stress relief system of claim 1, wherein said extended members include a cushioned exterior portion.

5. The back stress relief system of claim 1, wherein said leg cuffs are comprised of straps.

6. The back stress relief system of claim 1, wherein said leg cuffs are comprised of C-shaped locking members.

7. The back stress relief system of claim 1, wherein said sliding member is slidably positioned upon a lip portion within said support frame.

8. The back stress relief system of claim 1, wherein said drive unit comprises:
a hydraulic drive unit attached to said support frame; and
an elongate shaft attached between said hydraulic drive unit and said sliding member.

9. The back stress relief system of claim 8, including an adjustable extension member adjustably positioned between said elongate shaft and said sliding member.

10. The back stress relief system of claim 9, including a first sleeve attached to a cross member of said support frame for slidably receiving and supporting said elongate shaft.

11. The back stress relief system of claim 1, wherein said drive unit comprises:
an elongate bar member slidably positioned within said support frame, wherein said elongate bar includes a plurality of gears within one side thereof;
a wheel member rotatably attached to said support frame and attached to a shaft member;
a first drive sprocket attached to said shaft member opposite of said wheel member, wherein said first drive sprocket mechanically engages said plurality of gears within said elongate bar; and
an elongate shaft attached between said elongate bar member and said sliding member.

12. The back stress relief system of claim 11, including an adjustable extension member adjustably positioned between said elongate shaft and said sliding member.

13. The back stress relief system of claim 12, including a first sleeve attached to a cross member of said support frame for slidably receiving and supporting said elongate shaft.

14. The back stress relief system of claim 1, wherein said drive unit comprises:
an elongate bar member slidably positioned within said support frame, wherein said elongate bar includes a plurality of gears within one side thereof;
a drive motor attached to said support frame and mechanically connected to a shaft member;
a first drive sprocket attached to said shaft member opposite of said wheel member, wherein said first drive sprocket mechanically engages said plurality of gears within said elongate bar; and
an elongate shaft attached between said elongate bar member and said sliding member.

15. The back stress relief system of claim 14, including an adjustable extension member adjustably positioned between said elongate shaft and said sliding member.

16. The back stress relief system of claim 15, including a first sleeve attached to a cross member of said support frame for slidably receiving and supporting said elongate shaft.

17. The back stress relief system of claim 14, wherein said drive motor is comprised of an electric motor.
